(19) United States
(12) Patent Application Publication
(10) Pub. No.: US 2023/0133169 A1
(43) Pub. Date: May 4, 2023

(54) EGFR INHIBITOR, COMPOSITION, AND METHOD FOR PREPARATION THEREOF

(71) Applicant: BETTA PHARMACEUTICALS CO., LTD, Hangzhou, Zhejiang (CN)

(72) Inventors: Xiangyong LIU, Beijing (CN); Changyong QIU, Beijing (CN); Guolong DU, Beijing (CN); Qichao SHEN, Beijing (CN); Mengqiang LIU, Beijing (CN); Haitong SHENG, Beijing (CN); Lieming DING, Hangzhou (CN); Jiabing WANG, Beijing (CN)

(73) Assignee: BETTA PHARMACEUTICALS CO., LTD, Hangzhou, Zhejiang (CN)

(21) Appl. No.: 17/768,807

(22) PCT Filed: Oct. 13, 2020
(86) PCT No.: PCT/CN2020/120611

§ 371 Date: Apr. 13, 2022

(30) Foreign Application Priority Data
Oct. 17, 2019 (CN) PCT/CN2019/111636

(51) Int. Cl. C07D 239/48; C07D 403/10

(52) U.S. Cl. CPC C07D 239/48; C07D 403/10

(57) ABSTRACT

Provided are the compounds of Formula I, a method for using these compounds as an EGFR inhibitor, and a pharmaceutical composition comprising the compounds. The compound is used for treatment, prevention or amelioration of diseases or conditions such as cancer or infection.

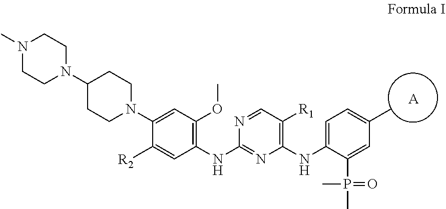

### FIELD OF THE INVENTION

The present invention is concerned with pharmaceutically active compounds, deuterated compounds (hydrogen substituted with deuterium) and pharmaceutically acceptable salts thereof, which can be used to treat or prevent diseases or medical conditions mediated by certain mutant forms of epidermal growth factor receptor (For example, L858R activating mutant, exon19 deletion activating mutant, T790M resistant mutant and C797S resistant mutant). The present invention also relates to a pharmaceutical composition comprising the compound, and a method of using the compound, deuterated compound and their relating salts to treat diseases mediated by various forms of EGFR mutants.

### BACKGROUND OF THE INVENTION

Epidermal growth factor receptor (EGFR) is a kind of transmembrane glycoprotein belonging to the ErbB family of tyrosine kinase receptors. Activation of EGFR leads to autophosphorylation of receptor tyrosine kinases, which are involved in the cascade of downstream signal transduction pathway that regulate cell proliferation, differentiation, and survival. EGFR is abnormally activated by various mechanisms, such as receptor overexpression, mutation, ligand-dependent receptor dimerization, ligand-independent activation, and is related to the development of a variety of human cancers.

EGFR inhibition is one of the key targets of cancer therapy. Despite the rapid development of previous generations of EGFR-Tkis, the problem of drug resistance has also emerged with the development of drugs. Most drug resistance is due to the T790M mutation of the ATP receptor. Recently developed third-generation irreversible inhibitors for T790M, such as osimertinib, have good activity on inhibitory, but drug resistance will inevitably appear. Most drug resistance is due to the T790M mutation of the ATP receptor. Recently developed third-generation irreversible inhibitors for T790M, such as osimertinib, have good activity on inhibitory, but drug resistance will inevitably appear. C797S mutation is a kind of missense mutation in which serine replaces cysteine at site 797 of exon 20 of EGFR, and C797S is located in the tyrosine kinase region of EGFR. C797S mutation prevents osimertinib from forming covalent bonds in the ATP binding domain, thus losing its inhibitory effect on EGFR activation and resulting in drug resistance.

Early patent applications WO2018108064, WO2018115218, WO2018181777 disclosed a series of fourth-generation EGFR inhibitors, but there is still a need for EGFR C797S inhibitors with stronger activity. In the invention, the applicant discovers a kind of small molecules that can be used as the fourth-generation EGFR inhibitor, whose activity can be used to treat cancer and/or infectious diseases. These small molecules are expected to be used as drugs with stability, solubility, bioavailability, therapeutic indices and toxicity values, which are essential to develop effective drugs for human health.

### SUMMARY OF INVENTION

The present invention relates to compounds capable of inhibiting EGFR which can be useful in the treatment of cancers and infectious diseases.

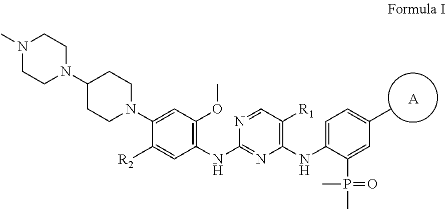

wherein,

R₁ is halogen, —C₁₋₆ alkyl or —C₁₋₆ alkoxy;

R₂ is selected from H, —C₁₋₆ alkyl, halogen or —C₅₋₆ heteroaryl, wherein heteroatom of —C₅₋₆ heteroaryl consists of one or two N, O, S atoms, and can be substituted with —C₁₋₆ alkyl;

ring A is selected from —C₃₋₆ saturated carbocycle or —C₃₋₆ saturated heterocycle, wherein the heteroatom of —C₃₋₆ saturated heterocycle consists of one or two N, O, S atoms.

For the compound of formula I, the present invention further provides some preferred technical solutions.

In some embodiments, R₁ is selected from Cl, Br or —OCH₃.

In some embodiments, R₁ is selected from Cl or Br.

In some embodiments, R₂ is selected from hydrogen, —CH₃, —CH₂CH₃,

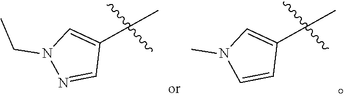

In some embodiments, R₂ is selected from —CH₂CH₃ or

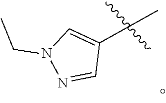 [SMILES: *c1cnn(CC)c1]

In some embodiments, R1 is selected from Br and R₂ is selected from —CH₂CH₃.

In some embodiments, ring A is selected from —C₃₋₆ saturated carbocycles, such as

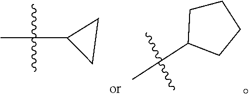

In some embodiments, ring A is selected from —C₃₋₆ saturated heterocycles, such as

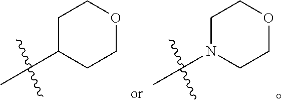 [SMILES: *C1CCOCC1 ; *N1CCOCC1]

The present invention provides the following specific compounds:

1) (2-((5-Bromo-2-((5-ethyl-2-methoxy-4-(4-(4-methylpiperazin-1-yl)piperidin-1-yl)phenyl)amino)pyrimidin-4-yl)amino)-5-cyclopropylphenyl)dimethylphosphine oxide;

2) (2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-cyclopropylphenyl)dimethylphosphine oxide;

3) (5-Cyclopropyl-2-((2-((5-(1-ethyl-1H-pyrazol-4-yl-2-methoxy-4-(4-(4-methylpiperazin-1-yl)piperidin-1-yl)phenyl)amino)-5-methoxypyrimidin-4-yl)amino)phenyl)dimethylphosphine oxide;

4) (2-((5-Chloro-2-((2-methoxy-4-(4-(4-methylpiperazin-1-yl)piperazin-1-yl)phenyl)amino)pyrimidin-4-yl)amino)-5-cyclopropylphenyl)dimethylphosphine oxide;

5) (5-propyl-2-((2-((5-ethyl-2-methoxy-4-(4-(4-methylpiperazin-1-yl)piperidin-1-yl)phenyl)amino)-5-methoxypyrimidin-4-yl)amino)phenyl)dimethylphosphine oxide;

6) (2-((5-Bromo-2-((5-ethyl-2-methoxy-4-(4-(4-methylpiperazin-1-yl)piperidin-1-yl)phenyl)amino)pyrimidin-4-yl)amino)-5-(tetrahydro-2H-pyran-4-yl)phenyl) dimethylphosphine oxide;

7) (2-((5-Chloro-2-((5-ethyl-2-methoxy-4-(4-(4-methylpiperazin-1-yl)piperidin-1-yl)phenyl)amino)pyrimidin-4-yl)amino)-5-cyclopentylphenyl)dimethylphosphine oxide;

8) (2-((5-Bromo-2-((5-ethyl-2-methoxy-4-(4-(4-methylpiperazin-1-yl)piperidin-1-yl)phenyl)amino)pyrimidin-4-yl)amino-5-morpholinyl)dimethylphosphine oxide;

9) (2-((5-Bromo-2-((2-methoxy-5-(1-methyl-1H-pyrrol-3-yl)-4-(4-(4-methylpiperazin-1-yl)piperidin-1-yl)phenyl)amino)pyrimidin-4-yl)-amino) cyclopropylphenyl)dimethylphosphine oxide; or

10) (2-((5-Bromo-2-((2-methoxy-5-methyl-4-(4-(4-methylpiperazin-1-yl)piperidin-1-yl)phenyl)amino)pyrimidin-4-yl)amino)-5-cyclopropylphenyl) dimethylphosphine oxide.

The compound of Formula I, or a stereoisomer, tautomer, deuterated compound, pharmaceutically acceptable salt, prodrug, chelate, non-covalent complex or solvate thereof.

The present invention provides a pharmaceutical composition which herein includes the compound of present invention, a pharmaceutically acceptable salt or stereoisomer thereof, and at least one pharmaceutically acceptable carrier or excipient.

The present invention further provides methods for inhibiting various forms of EGFR, including L858R, Δ19del, T790M and C797S, said method comprising administering to a patient any one of the compounds shown by the present invention, a pharmaceutically acceptable salt or stereoisomer thereof.

The present invention also provides methods for treating EGFR-driven cancer, said method comprising administering to a patient in need thereof a therapeutically effective amount of any of the compounds shown by the present invention, a pharmaceutically acceptable salt or stereoisomer thereof.

In some embodiments, the EGFR-driven cancer is characterized by the presence of one or more mutations from: (i) C797S, (ii) L858R and C797S, (iii) C797S and T790M, (iv) L858R, T790M and C797S, or (v) Δ19del, T790M and C797S.

In some implementations, the EGFR-driven cancers involve colon cancer, stomach cancer, thyroid cancer, lung cancer, leukemia, pancreatic cancer, melanoma, brain cancer, kidney cancer, prostate cancer, ovarian cancer, or breast cancer.

In some implementations, the mentioned lung cancer is non-small cell lung cancer caused by EGFR^{L858R/T790M/C797S} or EGFR^{Δ19del/T790M/C797S} mutant.

The present invention provides a method for inhibiting mutant EGFR in a patient, said method comprising administering to a patient in need thereof a therapeutically effective amount of the compounds shown by the present invention, a pharmaceutically acceptable salt or stereoisomer thereof.

The present invention provides the use in preparation of medicines for the compounds shown by the present invention or pharmaceutical composition thereof.

In some implementations, wherein the mentioned drugs are used to treat or prevent cancer.

In some implementations, wherein the cancers involve colon cancer, stomach cancer, thyroid cancer, lung cancer, leukemia, pancreatic cancer, melanoma, brain cancer, kidney cancer, prostate cancer, ovarian cancer, or breast cancer.

In some implementations, the mentioned lung cancer is non-small cell lung cancer caused by EGFRL^{858R/T790M/C797S} or EGFR^{Δ19del/T790M/C797S} mutant.

The general chemical terms used in the formula above have their usual meanings. For example, the term “halogen”, as used herein, unless otherwise indicated, means fluoro, chloro, bromo or iodo. The preferred halogen groups include F, Cl and Br.

Unless otherwise indicated, alkyl includes saturated monovalent hydrocarbon radicals having straight, branched chain or cyclic moieties. For example, alkyl include methyl, ethyl, propyl, isopropyl, cyclopropyl, n-butyl, isobutyl, sec-butyl, t-butyl, cyclobutyl, n-pentyl, 3-(2-methyl)butyl, 2-pentyl, 2-methylbutyl, neopentyl, cyclopentyl, n-hexyl, 2-hexyl, 2-methylpentyl and cyclohexyl. Similarly, C₁₋₈, as in C₁₋₈ alkyl is defined to identify the group as having 1, 2, 3, 4, 5, 6, 7 or 8 carbon atoms in a straight or branched arrangement.

Alkoxy radicals are oxygen ethers formed from the previously described straight, branched chain or cyclic alkyl.

Unless otherwise indicated, the term “aromatic ring” in the present invention refers to substituted or unsubstituted mono-, bis- or polycyclic aromatic groups comprising carbon atoms, or substituted or unsubstituted mono-, bis- or polycyclic aromatic groups comprising heteroatoms, such as N, O or S; when it is biscyclic or polycyclic, at least one ring is aromatic. Preferred aromatic ring is 5- to 10-membered monocycle or biscycle. Examples of aromatic rings include, but are not limited to phenyl, pyridyl, pyrazolyl, triazole, thiazole, furan, pyrimidinyl, pyrazinyl, pyrazolopyrimidine, benzodihydrofuran, pyrazolopyridine, benzoxazole.

Unless otherwise indicated, the term “heteroaryl”, as used herein, represents an unsubstituted or substituted stable 5- or 6-membered monocyclic aromatic ring system. It consists of preferred carbon atoms and one to four heteroatoms selected from N, O or S, and wherein the nitrogen or sulfur heteroatoms may optionally be oxidized, and the nitrogen heteroatom may optionally be quaternized. The heteroaryl group may be attached at any heteroatom or carbon atom which results in the creation of a stable structure. Examples of heteroaryl groups include, but are not limited to thienyl, furanyl, isoxazolyl, oxazolyl, pyrazolyl, pyrrolyl, thiazolyl, thiadiazolyl, triazolyl, pyridyl, pyridazinyl, indolyl.

The term “cycloalkyl” refers to a cyclic saturated alkyl chain with 3-12 carbon atoms, such as cyclopropyl, cyclobutyl, cyclobutyl, cyclobutyl.

The term “substituted” refers to a group in which one or more hydrogen atoms are each independently substituted with the same or different substituents. Typical substituents include but are not limited to halogen (F, Cl, Br or I), C₁₋₈ alkyl, C₃₋₁₂ cycloalkyl, —OR¹, —SR¹, ═O, ═S, —C(O)R¹, —C(S)R¹, ═NR¹, —C(O)OR¹, —C(S)OR¹, —NR¹R², —C(O)NR¹R², cyano, nitro, —S(O)₂R¹, —OS(O₂)OR¹, —OS(O)₂R¹, —OP(O)(OR¹)(OR²), wherein R¹ and R² are independently selected from —H, lower alkyl, and lower halogenated alkyl. In some embodiments, the substituents are independently selected from —F, —Cl, —Br, —I, —OH, trifluoromethoxy, ethoxy, propoxy, isopropoxy, n-butoxy, Isobutoxy, t-butoxy, —SCH₃, —SC₂H₅, formyl, —C(OCH₃), cyano, nitro, CF₃, —OCF₃, amino, dimethylamino, methylthio, sulfonyl and acetyl.

The term “composition”, as used herein, is intended to encompass a product comprising the specific ingredients in the specific amounts, as well as any product which results, directly or indirectly, from combinations of the specific ingredients in the specific amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

Embodiments of substituted alkyl include, but are not limited to, 2-aminoethyl, 2-hydroxyethyl, pentachloroethyl, trifluoromethyl, methoxymethyl, pentafluoroethyl, and piperazinylmethyl.

Embodiments of substituted alkoxy include, but are not limited to, aminomethoxy, tetrafluoromethoxy, 2-diethylaminoethoxy, 2-ethoxycarbonylethoxy, 3-hydroxypropoxy.

The compounds of the present invention may also present in the form of pharmaceutically acceptable salts. For use in medicine, the salts of the compounds in this invention refer to a non-toxic “pharmaceutically acceptable salts”. The forms of pharmaceutically acceptable salts include pharmaceutically acceptable acidic/anionic or basic/cationic salts. The pharmaceutically acceptable acid/anionic salts usually take the form of protonation of inorganic or organic acid for basic nitrogen. Representative organic or inorganic acids include hydrochloric acid, hydrobromic acid, hydroiodic acid, perchloric acid, sulfuric acid, nitric acid, phosphoric acid, acetic acid, propionic acid, glycolic acid, lactic acid, succinic acid, maleic acid, fumaric acid, apple Acid, tartaric acid, citric acid, benzoic acid, mandelic acid, methanesulfonic acid, oxyethylsulfonic acid, benzenesulfonic acid, oxalic acid, pamoic acid, 2-naphthalenesulfonic acid, p-toluenesulfonic acid, cyclohexane sulfamic acid, Salicylic acid, saccharin or trifluoroacetic acid. Pharmaceutically acceptable alkaline/cationic salts include, but are not limited to, aluminum, calcium, chloroprocaine, choline, diethanolamine, ethylenediamine, lithium, magnesium, potassium, sodium, and zinc.

The present invention includes within its scope the prodrugs of the compounds of this invention. Generally, the prodrugs refer to functional derivatives that are easily converted in vivo into desired compounds. Therefore, in the therapeutic method of the present invention, the term “administration” shall include the treatment of various conditions described for specific disclosed compounds, or the use of compounds that may not be specifically disclosed, but are converted into specific compounds in vivo after administration. Conventional methods for selecting and preparing suitable derivatives of prodrugs have been documented in books Design of Prodrugs (ed. H. Bundgaard, Elsevier, 1985), etc.

Obviously, the definition of any substituent or variable at a particular location in a molecule is independent of any other position in the molecule. It is easy to understand that the substituent or substituted patterns of the compound of the present invention can be selected by ordinary technicians to provide compounds that are chemically stable and that can be readily synthesized by techniques know in the art as well as those methods set forth herein.

The present invention includes compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The above Formula I are shown without a definitive stereochemistry at certain positions. The present invention includes all stereoisomers of the compounds shown by Formula I and their pharmaceutically acceptable salts. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound shown by Formula I exists, the present invention includes any possible tautomers and pharmaceutically acceptable salts thereof, and mixtures thereof, except where specifically stated otherwise.

When the compound of Formula I and pharmaceutically acceptable salts thereof exist in the form of solvates with water or polymorphic forms, the present invention includes any possible solvates and polymorph. A type of a solvent that forms the solvate with water is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids, particularly preferred are formic and hydrochloric acid. Since the compounds of Formula I are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or a prodrug, or a metabolite, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently prepared into the desired appearance.

Thus, the pharmaceutical compositions of this invention may include a pharmaceutically acceptable carrier compounds shown by Formula I or a tautomers, polymorphs, solvates, pharmaceutically acceptable salts and prodrugs thereof. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include such as lactose, terra alba, sucrose, talc, gelatin, agar, pectin, acacia, magnesium stearate, and stearic acid. Examples of liquid carriers include such as sugar syrup, peanut oil, olive oil, and water. Examples of gaseous carriers include such as carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05mg to about 5g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier (s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, inflammation, cancer, psoriasis, allergy/asthma, diseases and discomforts of the immune system, and diseases and discomforts of the central nervous system (CNS), are effectively treated at the dosage levels of the drug ranging from about 0.01 to 50 mg of the compound per kilogram of body weight per day, or alternatively about 0.5 mg to about 3.5 g per patient per day.

It is understood, however, that lower or higher doses than those recited above may be required. Specific dose level and treatment regimens for any particular subject will depend upon a variety of factors, including the activity of the specific compound employed, the age, body weight, general health, sex, diet, time of administration, route of administration, rate of excretion, drug combination, the severity and course of the particular disease undergoing therapy.

These and other aspects will become apparent from the following written description of the invention.

The following Examples are provided to better illustrate the present invention. All parts and percentages are by weight and all temperatures are degrees Celsius, unless explicitly stated otherwise.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. According to at least one of the assay methods described herein, the compounds of the embodiments have been found to have the ability to inhibit L858R, Δ 19del, T790M and C797S.

### Embodiment

It should be understood that the preceding general descriptions and the detailed descriptions below are exemplary and illustrative only and are not a restriction on any subject requiring protection. Unless expressly stated otherwise, all portions and percentages are by weight, and all temperature units are in degrees Celsius. The compounds described herein can be obtained from commercial sources or synthesized by conventional methods as shown below using commercially available raw materials and reagents.

The following abbreviations have been used in embodiments:

DIEA: N,N-diisopropylethylamine;

DMF: N,N-dimethylformamide;

DMSO: Dimethyl sulfoxide;

HEPES: 4-(2-hydroxyethyl)-1-piperazine ethanesulfonic acid;

LCMS: Liquid Chromatography-Mass Spectrometry;

h or hrs: hours;

Pd/C: Palladium on active carbon;

Pd(dppf)Cl₂: [1,1′-Bis(diphenylphosphino)ferrocene]dichloropalladium;

MeOH: Methanol;

TLC: Thin Layer Chromatography;

Xantphos: 4,5-Bis(diphenylphosphino)-9,9-dimethylxanthene;

Pd(OAc)₂: Palladium acetate;

TsOH: p-toluenesulfonic acid;

n-BuOH: n-butanol.

### EXAMPLE 1

### Synthesis of Compound 1

### (2-((5-Bromo-2-((5-ethyl-2-methoxy-4-(4-(4-methylpiperazin-1-yl)piperidin-1-yl)phenyl)amino)pyrimidin-4-yl)amino)-5-cyclopropylphenyl)dimethylphosphine oxide

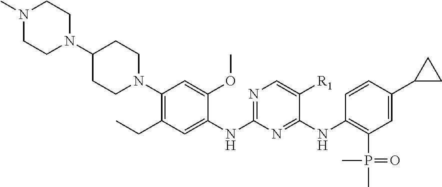

### Step 1: Synthesis of Compound 1-3

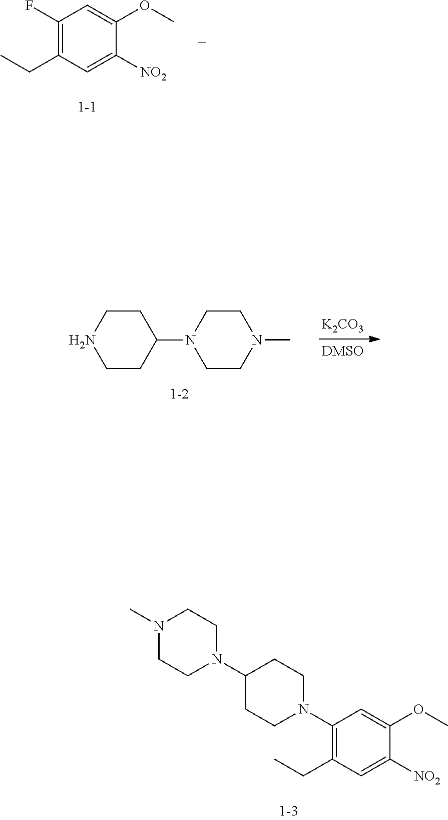

Add 1-1 (3.00 g), 1-2 (4.14 g), K₂CO₃ (6.24 g) and DMSO (30 mL) successively to the reaction flask, raise temperature to 90° C. and stir with heating for 12 hr. The reaction was complete as monitored by LCMS, and then stopped. The reaction solution was poured into water (100 mL) and filtered with suction. The filter cake was washed with water, and dried to obtain the yellow solid of desired product 1-3 (4.20 g), MS: 363 [M+H]⁺

### Step 2: Synthesis of Compound 1-4

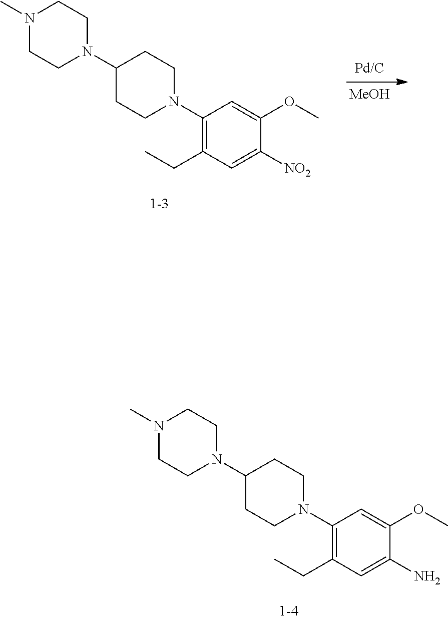

Add compound 1-3 (4.20 g), Pd/C (1.00 g) and MeOH (60 mL) successively to the reaction flask, introduce H₂. The reaction solution was stirred at room temperature for 3 hr. The reaction was complete as monitored by LCMS, and then stopped. The solution was filtered with suction and rinsed with methanol (20 mL); the organic phase was collected, and the solvent was removed to obtain the reddish-brown liquid of desired compound 1-4 (3.5 g), MS: 333[M+H]⁺

### Step 3: Synthesis of Compound 1-6

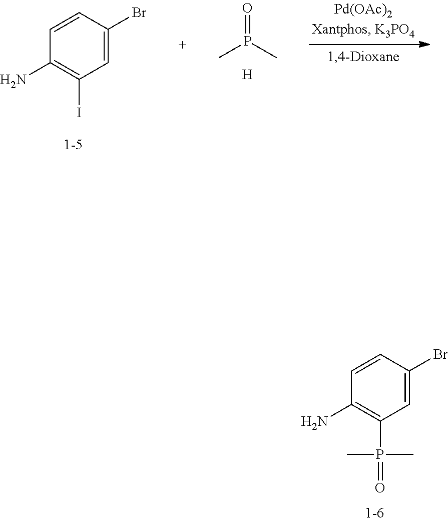

Add compound 1-5 (3.0 g), dimethylphosphine oxide (865 mg), K₃PO₄ (6.41 g), Pd(OAc)₂ (226 mg), Xantphos (1.75 g), 1,4-Dioxane (60 mL) successively to the reaction flask, heat to 100° C. under nitrogen protection, stir with heating for 4 hr. The reaction was complete as monitored by LCMS, and then stopped. The water was added to the reaction solution, and which was extracted with dichloromethane (3×50 mL), the organic phase was washed with saturated brine (3×30 mL), dried over anhydrous sodium sulfate, separated and dried by column chromatography (dichloromethane:methanol=15:1); the solvent was removed to obtain the yellow solid of desired product 1-6 (2.0 g), MS: 248[M+H]⁺

### Step 4: Synthesis of Compound 1-8

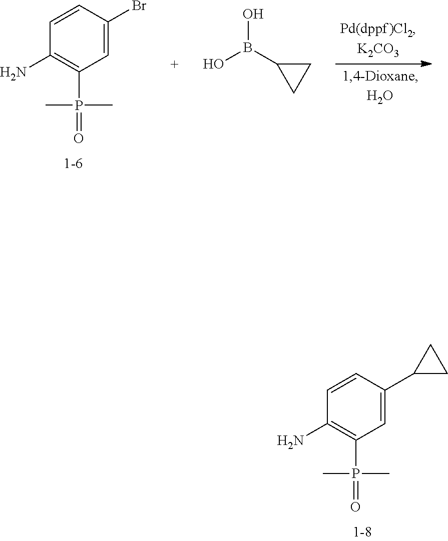

Add compounds 1-6 (2.20 g), 1-7 (1.52 g), K₃PO₄ (5.65 g), Pd(dppf)Cl₂ (649 mg), 1,4-dioxane (30 mL) and water (3 mL) successively to the reaction flask, heat to 100° C. under nitrogen protection, stir with heating for 12 hr. The reaction was complete as monitored by LCMS, and then stopped. The water (50 mL) was added to the reaction solution, and which was extracted with dichloromethane (3×50 mL), the organic phase was washed with saturated brine (3×30 mL), dried over anhydrous sodium sulfate, separated and dried by column chromatography (dichloromethane:methanol=10:1); the solvent was removed to obtain the brown solid of desired product 1-8 (1.20 g), MS: 210[M+H]⁺

### Step 5: Synthesis of Compound 1-10

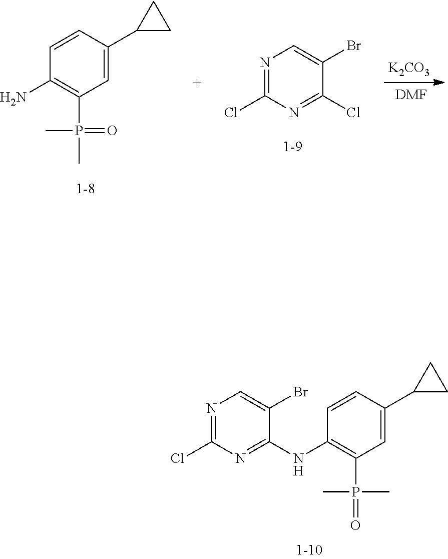

Add compounds 1-8 (364 mg), 1-9 (794 mg), K₂CO₃ (721 mg) and DMF (10 mL) successively to the reaction flask, heat to 100° C., stir with heating for 12 hr. The reaction was complete as monitored by LCMS, and then stopped. The water (50 mL) was added to the reaction solution, and which was extracted with dichloromethane (3×50 mL), the organic phase was washed with saturated brine (3×30 mL), dried over anhydrous sodium sulfate, separated and dried by column chromatography (dichloromethane:methanol=13:1); the solvent was removed to obtain the yellowish solid of desired product 1-10 (330 mg), MS: 400 [M+H]⁺

### Step 6: Synthesis of Compound 1

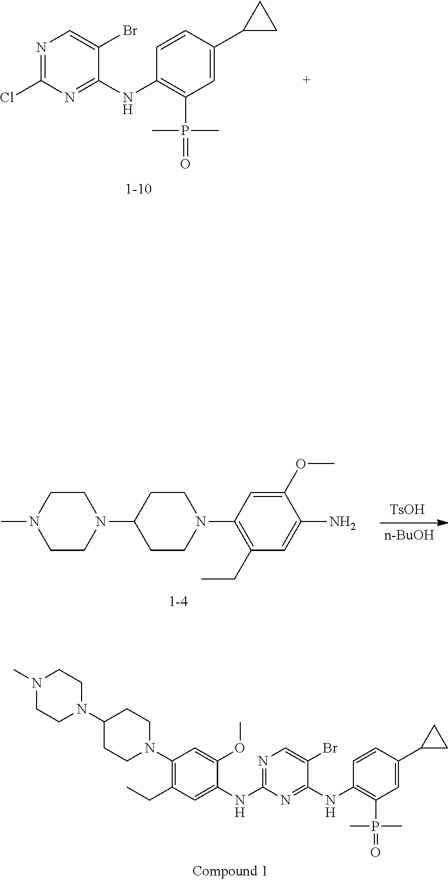

Add compounds 1-10 (80 mg), 1-4 (80 mg), p-toluenesulfonic acid (52 mg) and n-butanol (2 mL) successively to the reaction flask, heat to 100° C., stir with heating for 12 hr. The reaction was complete as monitored by LCMS, and then stopped. The reaction solution was poured into 2N sodium carbonate solution (30 mL), extracted with mixed solvent of dichloromethane/methanol=10/1 (3×30 mL), combined with organic phase, washed with saturated brine (3×30 mL), dried over anhydrous sodium sulfate and concentrated. The residue was separated and purified by thick preparative plate (dichloromethane:methanol=12:1), and the eluted product was concentrated to obtain the off-white solid compound 1 (25.5 mg). MS: 696[M+H]⁺, ¹H NMR (500 MHz, DMSO) δ 10.708(s, 1H), 8.186-8.176(m, 1H), 8.143-8.111(m, 1H,), 8.030(s, 1H), 7.407 (s, 1H), 7.281 (d, 1H, J=14), 6.920 (d, 1H, J=9), 6.768 (s, 1H), 3.7463 (s, 3H), 3.013 (m, 2H), 2.715 (m, 2H), 2.603 (m, 1H), 2.362 (m, 8H), 2.178 (s, 3H), 1.904 (m, 3H), 1.755 (d, 6H, J=13), 1.592 (m, 2H), 1.235 (s, 2H), 1.037(m, 3H), 0.959 (m, 2H), 0.651 (m, 2H).

### EXAMPLE 2

### Synthesis of Compound 2

### 2-((5-Bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-cyclopropylphenyl)dimethylphosphine oxide

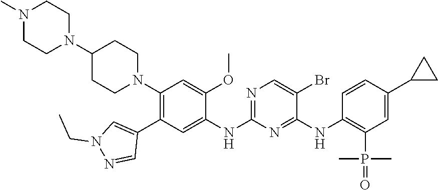 [SMILES: CCn1cc(-c2cc(Nc3ncc(Br)c(Nc4ccc(C5CC5)cc4P(C)(C)=O)n3)c(OC)cc2N2CCC(N3CCN(C)CC3)CC2)cn1]

### Step 1: Synthesis of Compound 2-3

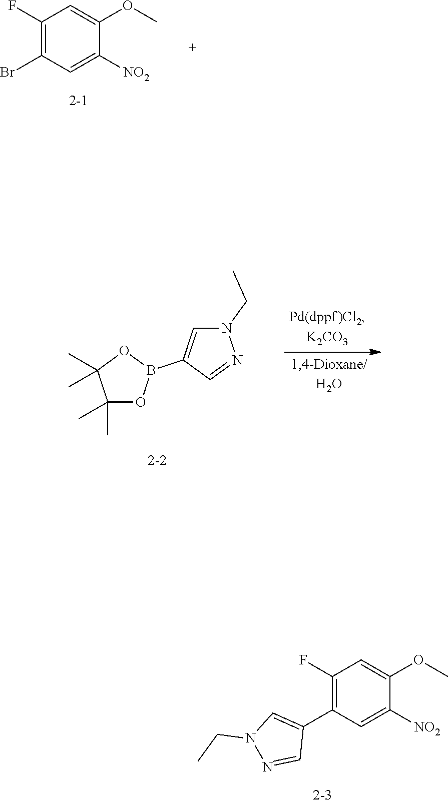 [SMILES: COc1cc(F)c(Br)cc1[N+](=O)[O-] ; CCn1cc(B2OC(C)(C)C(C)(C)O2)cn1 ; CCn1cc(-c2cc([N+](=O)[O-])c(OC)cc2F)cn1]

Dissolve compound 2-1 (1.50 g) in 1,4-dioxane (10 ml) and water (2 ml) in a 50 ml stand-up bottle, and then add compound 2-2 (1.60 g), Pd (dppf)Cl₂ (489.94 mg) and anhydrous potassium carbonate (995.03 mg). The reaction system was heated to 100° C. under nitrogen protection and stirred overnight. It was naturally cooled down to room temperature. Add ethyl acetate/water (30 ml/30 ml) for layering, collect organic phase and concentrate. The residue mixed with silica gel, and purified by flash silica gel column (Phase A: n-hexane, phase B: ethyl acetate; B%: 0-100%, 20 min); the product eluent was collected and concentrated to obtain the compound 2-3 (1.20 g) MS: 266 [M+H]⁺

### Step 2: Synthesis of Compound 2-4

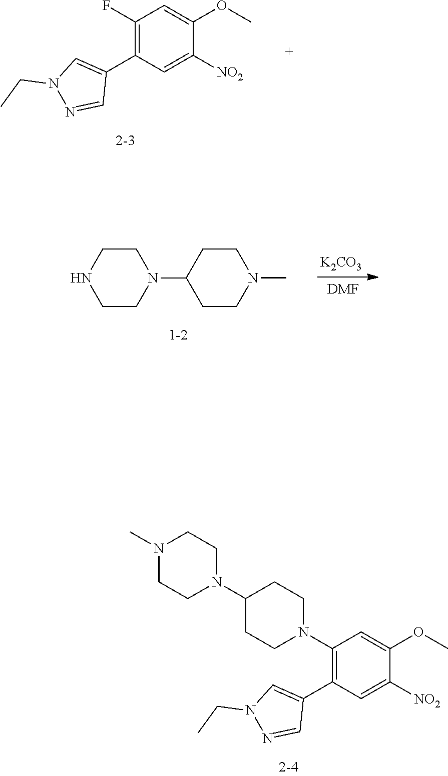

The compound 2-4 was synthesized as the method described for compound 1-3 using compound 2-3 instead of raw compound 1-1. For compound 2-4, MS: 429[M+H]⁺

### Step 3: Synthesis of Compound 2-5

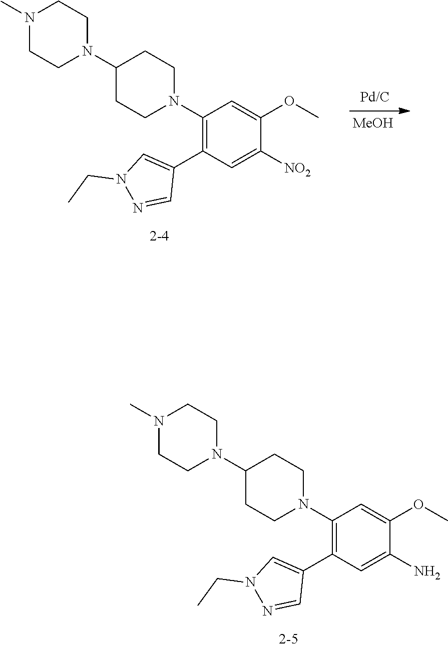

The compound 2-5 was synthesized as the method described for compound 1-4 using compound 2-4 instead of raw compound 1-3. For compound 2-5, MS: 399[M+H]⁺

### Step 4: Synthesis of Compound 2

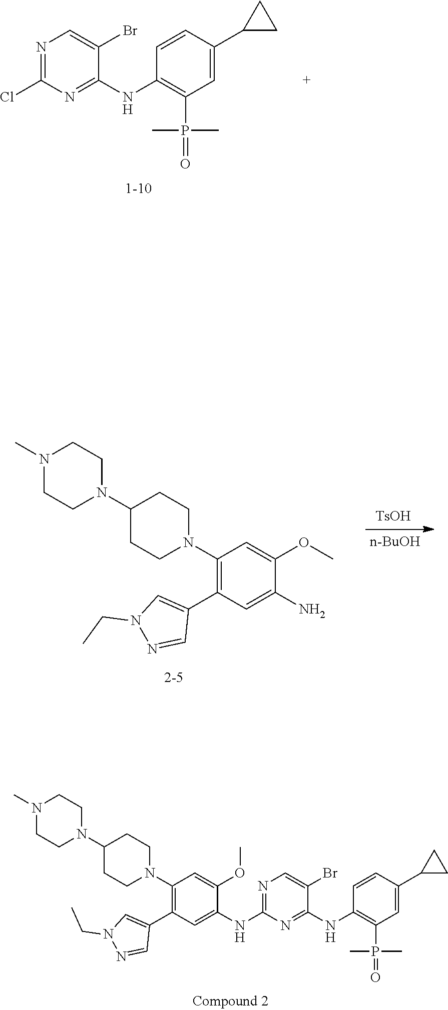

The compound 2 was synthesized as the method described for compound 1 using compound 2-5 instead of raw compound 1-4. For compound 2, MS: 762[M+H]⁺

### EXAMPLE 3

### Synthesis of Compound 3

### (5-Cyclopropyl-2-((2-((5-(1-ethyl-1H-pyrazol-4-yl)-2-methoxy-4-(4-(4-methylpiperazin-1-yl)piperidin-1-yl)phenyl)amino)-5-methoxypyrimidin-4-yl)amino)phenyl)dimethylphosphine oxide

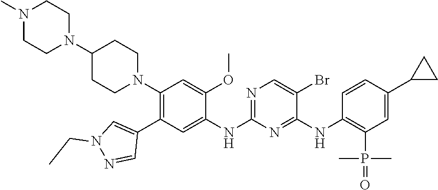 [SMILES: CCn1cc(-c2cc(Nc3ncc(Br)c(Nc4ccc(C5CC5)cc4P(C)(C)=O)n3)c(OC)cc2N2CCC(N3CCN(C)CC3)CC2)cn1]

### Step 1: Synthesis of Compound 3-2

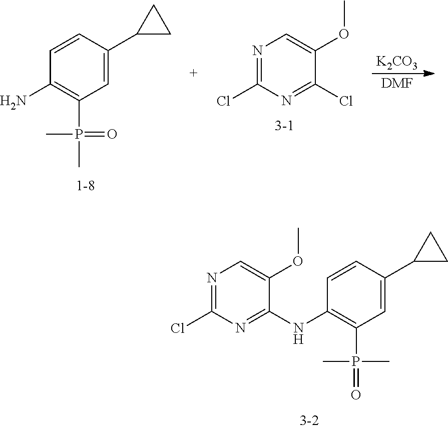

The compound 3-2 was synthesized as the method described for compound 1-10 using compound 3-1 instead of raw compound 1-9. For compound 3-2, MS: 352[M+H]⁺

### Step 2: Synthesis of Compound 3

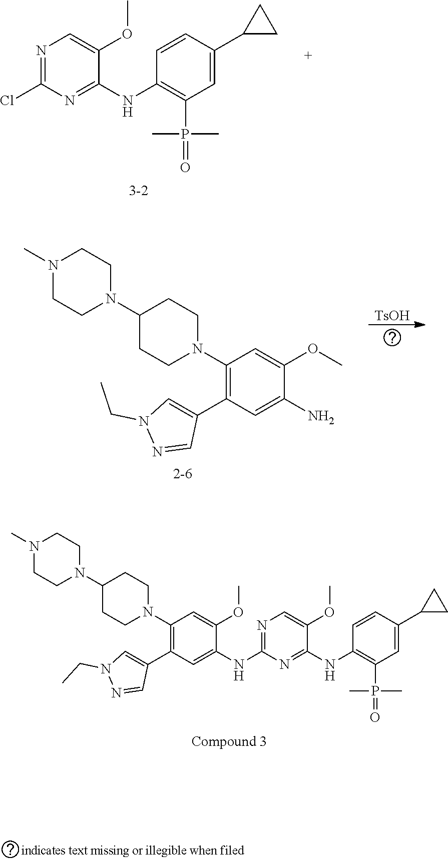

The compound 3 was synthesized as the method described for compound 1 using compound 3-2 instead of raw compound 1-10, compound 2-5 instead of raw compound 1-4. For compound 3, MS:714 [M+1-1]⁺

### EXAMPLE 4

### Synthesis of Compound 4

### (2-((5-Chloro-2-((2-methoxy-4-(4-(4-methylpiperazin-1-yl)piperazin-1-yl)phenyl)amino)pyrimidine-4-yl)amino)-5-cyclopropylphenyl)dimethylphosphine oxide

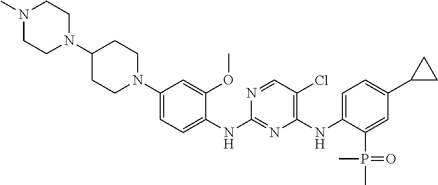 [SMILES: COc1cc(N2CCC(N3CCN(C)CC3)CC2)ccc1Nc1ncc(Cl)c(Nc2ccc(C3CC3)cc2P(C)(C)=O)n1]

### Step 1: Synthesis of Compound 4-2

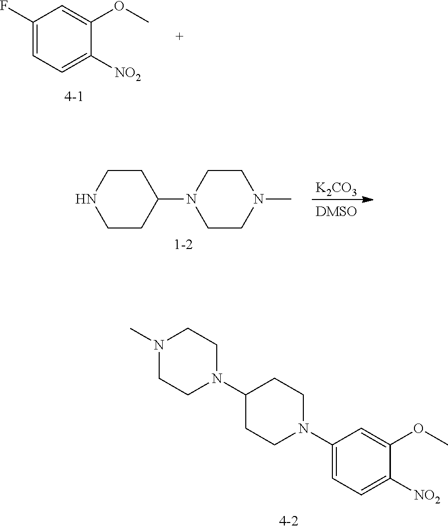

The compound 4-2 was synthesized as the method described for compound 1-3 using compound 4-1 instead of compound 1-1. For compound 4-2, MS: 335[M+H]⁺

### Step 2: Synthesis of Compound 4-3

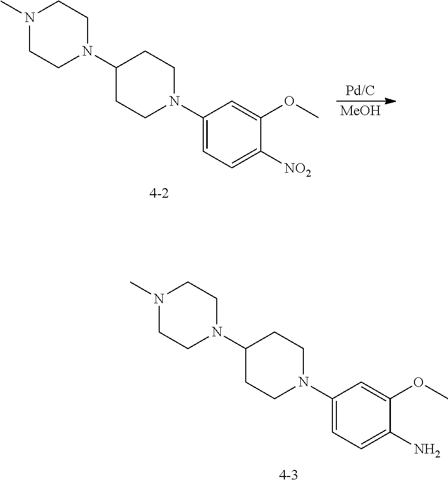

The compound 4-3 was synthesized as the method described for compound 1-4 using compound 4-2 instead of compound 1-3. For compound 4-3, MS: 305[M+H]⁺

### Step 3: Synthesis of Compound 4-5

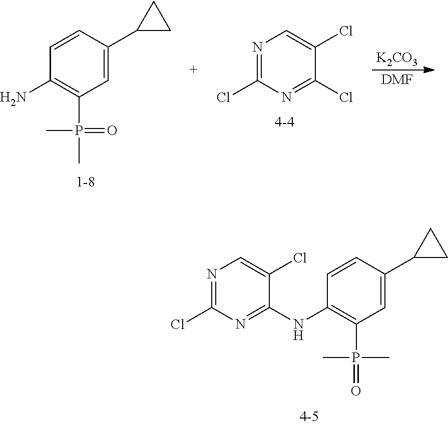

The compound 4-5 was synthesized as the method described for compound 1-10 using compound 4-4 instead of raw compound 1-9. For compound 4-5, MS: 356[M+H]⁺

### Step 3: Synthesis of Compound 4

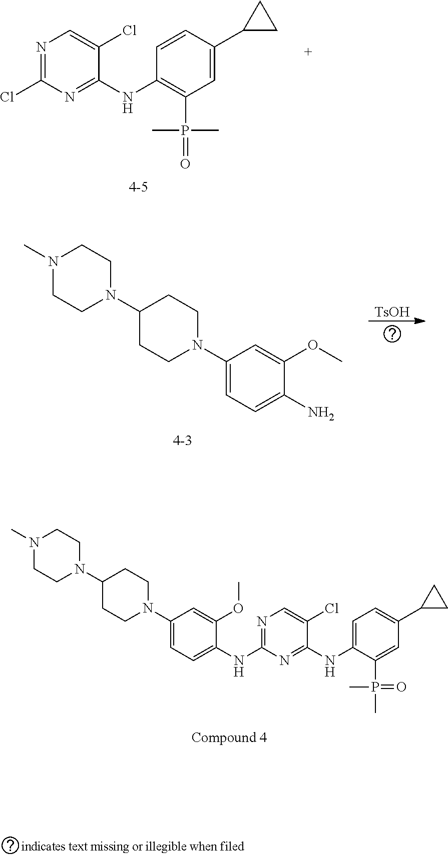

The compound 4 was synthesized as the method described for compound 1 using compound 4-5 instead of raw compound 1-10, compound 4-3 instead of raw compound 1-4. For compound 4, MS: 624 [M+H]⁺

### EXAMPLE 5

### Synthesis of Compound 5

### (5-propyl-2-((2-((5-ethyl-2-methoxy-4-(4-(4-methylpiperazin-1-yl)piperidin-1-yl)phenyl)amino)-5-methoxypyrimidin-4-yl)amino)phenyl)dimethylphosphine oxide

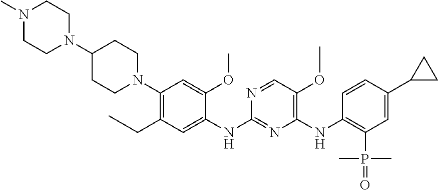 [SMILES: CCc1cc(Nc2ncc(OC)c(Nc3ccc(C4CC4)cc3P(C)(C)=O)n2)c(OC)cc1N1CCC(N2CCN(C)CC2)CC1]

### Step 1: Synthesis of Compound 5

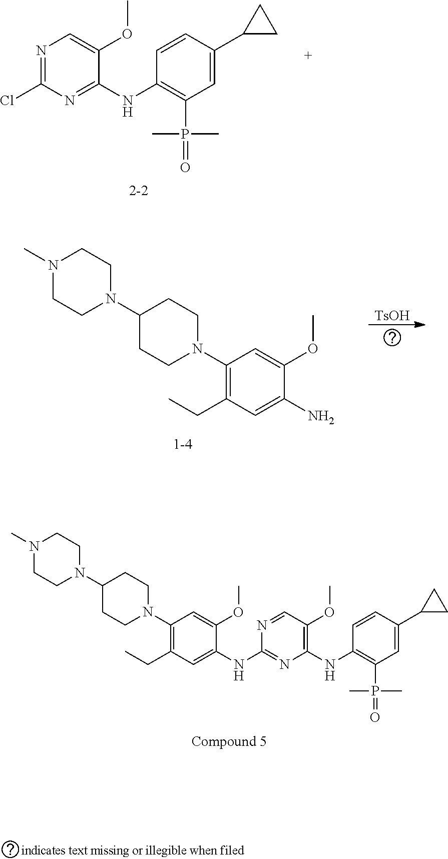 [SMILES: COc1cnc(Cl)nc1Nc1ccc(C2CC2)cc1P(C)(C)=O ; CCc1cc(N)c(OC)cc1N1CCC(N2CCN(C)CC2)CC1 ; "TsOH" ; CCc1cc(Nc2ncc(OC)c(Nc3ccc(C4CC4)cc3P(C)(C)=O)n2)c(OC)cc1N1CCC(N2CCN(C)CC2)CC1]

The compound 5 was synthesized as the method described for compound 1 using compound 2-2 instead of raw compound 1-10. For compound 5, MS: 648 [M+H]⁺

### EXAMPLE 6: Synthesis of Compound 6

### (2-((5-Bromo-2-((5-ethyl-2-methoxy-4-(4-(4-methylpiperazin-1-yl)piperidin-1-yl)phenyl)amino)pyrimidin-4-yl)amino)-5-(tetrahydro-2H-pyran-4-yl)phenyl)dimethylphosphine oxide

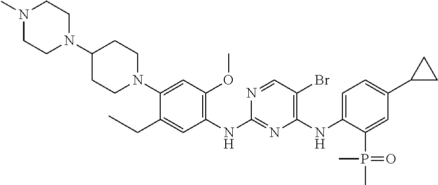 [SMILES: CCc1cc(Nc2ncc(Br)c(Nc3ccc(C4CC4)cc3P(C)(C)=O)n2)c(OC)cc1N1CCC(N2CCN(C)CC2)CC1]

### Step 1: Synthesis of Compound 6-1

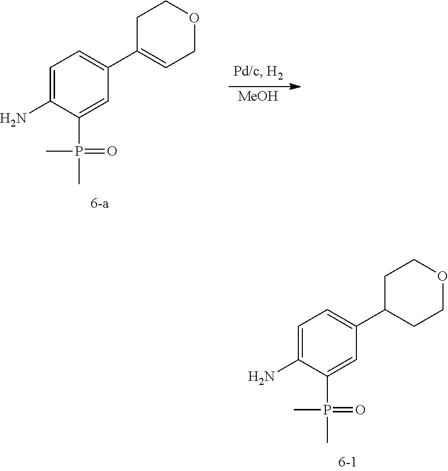 [SMILES: "MeOH" ; CP(C)(=O)c1cc(C2=CCOCC2)ccc1N ; CP(C)(=O)c1cc(C2CCOCC2)ccc1N]

Dissolve compound 6-a (400 mg) in 15 mL of methanol, add palladium/carbon (100 mg, 20%); the gas in the reactor was replaced with H₂ for three times, and the reaction was carried out for 2 hr at room temperature. After filtering, the solvent was removed to obtain the yellow solid 6-1 (300 mg), MS: 254 [M+H]⁺

### Step 2: Synthesis of Compound 6-2

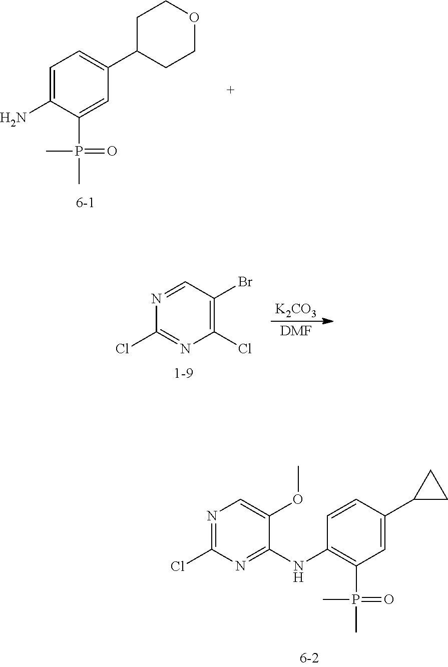

The compound 6-2 was synthesized as the method described for compound 1-10 using compound 6-1 instead of raw compound 1-8. For compound 6-2, MS: 444 [M+H]⁺

### Step 3: Synthesis of Compound 6

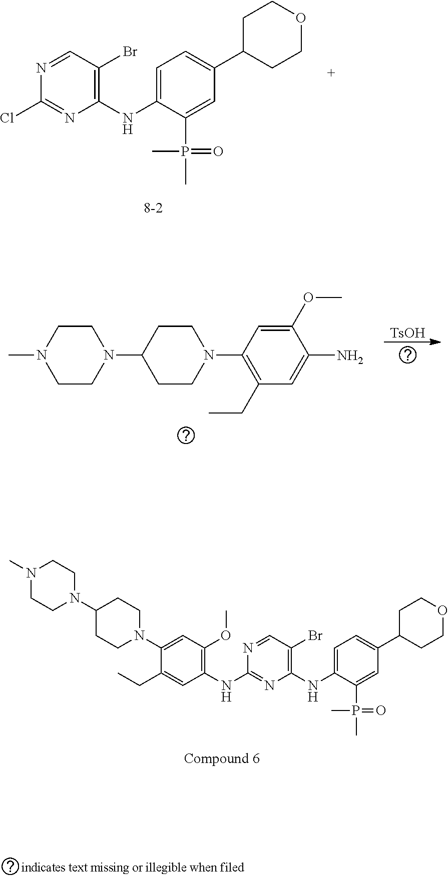

The compound 6 was synthesized as the method described for compound 1 using compound 6-2 instead of raw compound 1-10. For compound 6, MS: 740 [M+H]⁺

### EXAMPLE 7

### Synthesis of Compound 7

### (2-((5-Chloro-2-((5-ethyl-2-methoxy-4-(4-(4-methylpiperazin-1-yl)piperidin-1-yl)phenyl)amino)pyrimidin-4-yl)amino)-5-cyclopentylphenyl)dimethylphosphine oxide

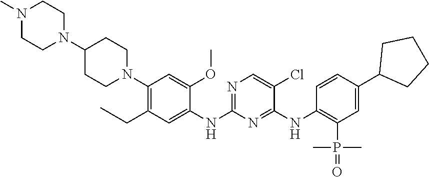 [SMILES: CCc1cc(Nc2ncc(Cl)c(Nc3ccc(C4CCCC4)cc3P(C)(C)=O)n2)c(OC)cc1N1CCC(N2CCN(C)CC2)CC1]

### Step 1: Synthesis of Compound 7-2

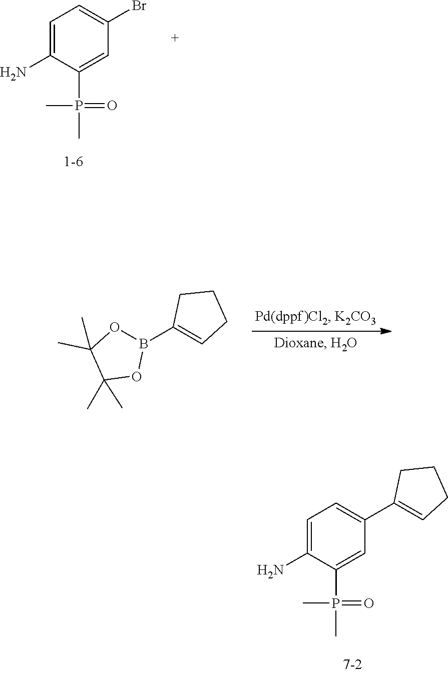 [SMILES: CP(C)(=O)c1cc(Br)ccc1N ; CC1(C)OB(C2=CCCC2)OC1(C)C ; CP(C)(=O)c1cc(C2=CCCC2)ccc1N]

The compound 7-2 was synthesized as the method described for compound 1-8 using compound 7-1 instead of raw compound 1-7. For compound 7-2, MS: 236 [M+H]⁺

### Step 2: Synthesis of Compound 7-3

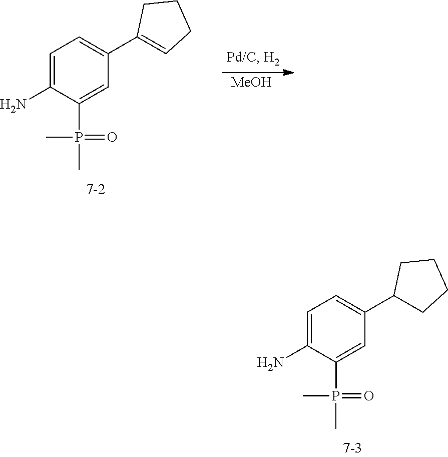 [SMILES: "MeOH" ; CP(C)(=O)c1cc(C2=CCCC2)ccc1N ; CP(C)(=O)c1cc(C2CCCC2)ccc1N]

The compound 7-3 was synthesized as the method described for compound 6-1 using compound 7-2 instead of raw compound 6-a. For compound 7-3, MS: 238 [M+H]⁺

### Step 3: Synthesis of Compound 7-4

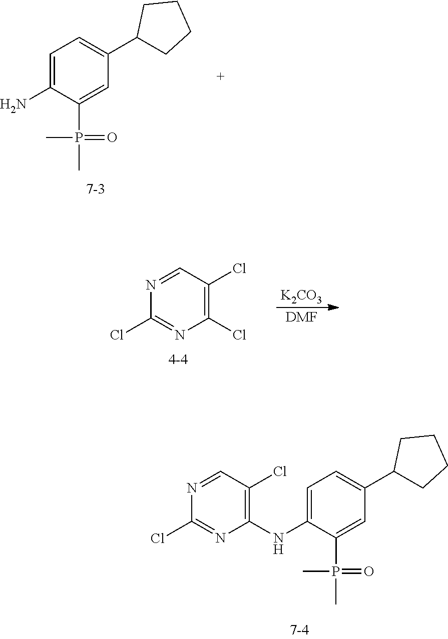

The compound 7-4 was synthesized as the method described for compound 1-10 using compound 7-3 instead of raw compound 1-8, compound 4-4 instead of raw compound 1-9. For compound 7-4, MS: MS: 384 [M+H]⁺

### Step 4: Synthesis of Compound 7

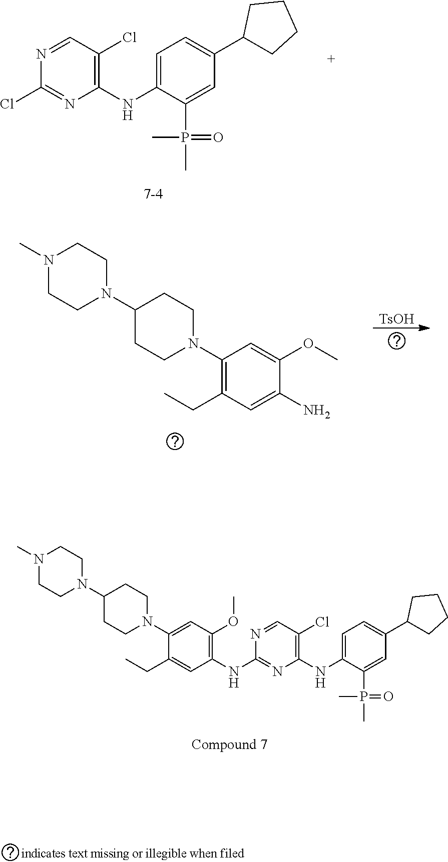

The compound 7 was synthesized as the method described for compound 1 using compound 7-4 instead of raw compound 1-10. For compound 7, MS: 680 [M+H]⁺

### EXAMPLE 8

### Synthesis of Compound 8

### (2-((5-Bromo-2-((5-ethyl-2-methoxy-4-(4-(4-methylpiperazin-1-yl)piperidin-1-yl)phenyl)amino)pyrimidin-4-yl)amino-5-morpholinyl)dimethylphosphine oxide

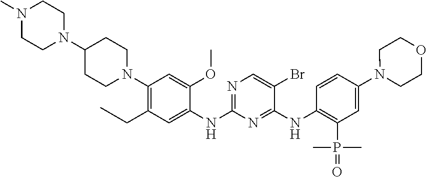 [SMILES: CCc1cc(Nc2ncc(Br)c(Nc3ccc(N4CCOCC4)cc3P(C)(C)=O)n2)c(OC)cc1N1CCC(N2CCN(C)CC2)CC1]

### Step 1: Synthesis of Compound 8-3

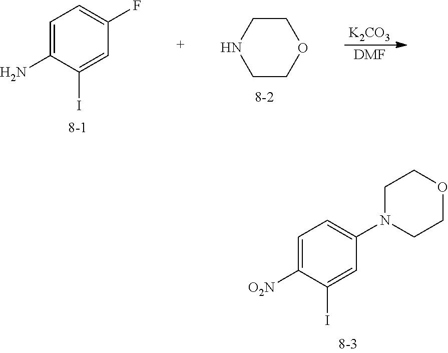

Dissolve 8-1 (267 mg), 8-2 (105 mg) and anhydrous potassium carbonate (415 mg) in DMF (5 mL) in a 50 mL stand-up bottle; the reaction solution was heated to 100° C. and stirred for 2 hr, cooled down to room temperature, diluted with water (30 ml), extracted twice with ethyl acetate (30 ml*2), combined with organic phase, washed three times with water (50 ml*3), dried and concentrated, and then separated and purified by column chromatography (dichloromethane:methanol=15:1) to obtain the compound 8-3 (313 mg), MS: 335 [M+H]⁺

### Step 2: Synthesis of Compound 8-4

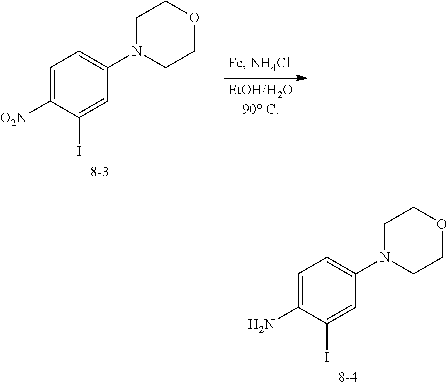 [SMILES: O=[N+]([O-])c1ccc(N2CCOCC2)cc1I ; Nc1ccc(N2CCOCC2)cc1I]

Dissolve 8-3 (313 mg) in absolute ethyl alcohol (10 mL) /H₂O (2 mL) in a 50 ml stand-up bottle, add iron powder (523 mg) and ammonium chloride (501 mg); the reaction solution was heated to 90° C. and stirred for 2 hr. After complete reaction, the reaction solution was cooled down to room temperature, and filtered by suction bottle with diatomite; the filter cake was rinsed with 50 ml of absolute ethyl alcohol; the filtrate was collected and concentrated; the residue was dissolved in DCM/H₂O (30 ml/30 ml); the organic phase was collected, dried over anhydrous sodium sulfate, filtered and concentrated to get 8-4 (224 mg), MS: 305 [M+H]⁺

### Step 3: Synthesis of Compound 8-5

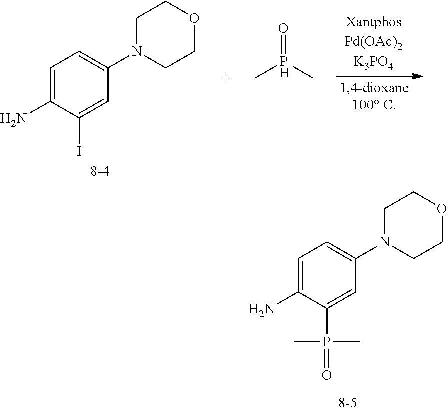

The compound 8-5 was synthesized as the method described for compound 1-6 using compound 8-4 instead of raw compound 1-5. For compound 8-5, MS:255 [M+H]⁺

### Step 4: Synthesis of Compound 8-6

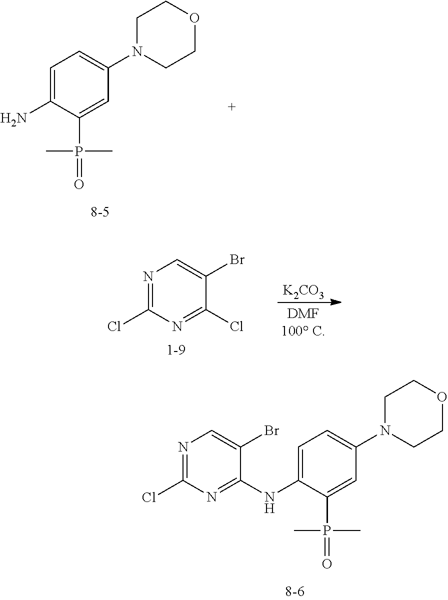

The compound 8-6 was synthesized as the method described for compound 1-10 using compound 8-5 instead of raw compound 1-8. For compound 8-6, MS:445 [M+H]⁺

### Step 5: Synthesis of Compound 8

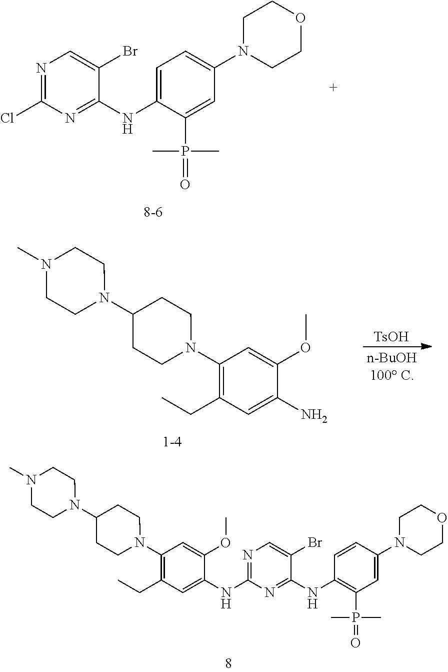 [SMILES: CP(C)(=O)c1cc(N2CCOCC2)ccc1Nc1nc(Cl)ncc1Br ; CCc1cc(N)c(OC)cc1N1CCC(N2CCN(C)CC2)CC1 ; "TsOH" ; CCc1cc(Nc2ncc(Br)c(Nc3ccc(N4CCOCC4)cc3P(C)(C)=O)n2)c(OC)cc1N1CCC(N2CCN(C)CC2)CC1]

The compound 8 was synthesized as the method described for compound 1 using compound 8-6 instead of raw compound 1-10. For compound 8, MS:741 [M+H]⁺

### EXAMPLE 9

### Synthesis of Compound 9

### (2-((5-Bromo-2-((2-methoxy-5-(1-methyl-1H-pyrrol-3-yl)-4-(4-(4-methylpiperazine-1-yl)piperidin-1-yl)phenyl)amino)pyrimidin-4-yl)-amino)-5-cyclopropylphenyl)dimethylphosphine oxide

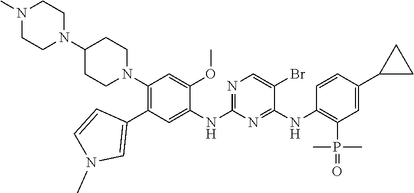 [SMILES: COc1cc(N2CCC(N3CCN(C)CC3)CC2)c(-c2ccn(C)c2)cc1Nc1ncc(Br)c(Nc2ccc(C3CC3)cc2P(C)(C)=O)n1]

### Step 1: Synthesis of Compound 9-2

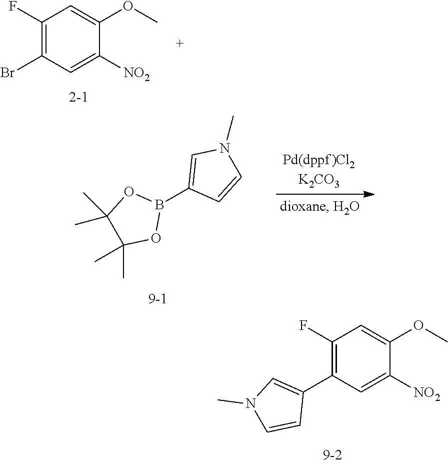

The compound 9-2 was synthesized as the method described for compound 2-3 using compound 9-1 instead of raw compound 2-2. For compound 9-2, MS: 251[M+H]⁺

### Step 2: Synthesis of Compound 10-3

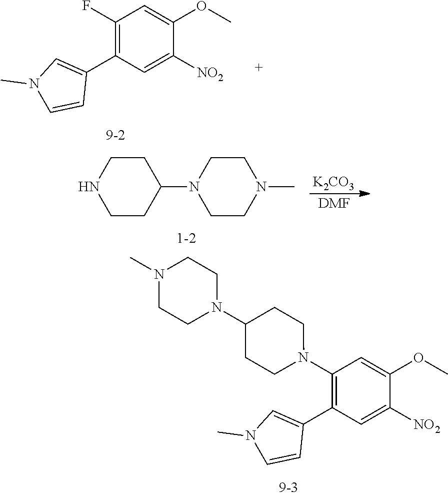

The compound 9-3 was synthesized as the method described for compound 1-3 using compound 9-2 instead of raw compound 1-1. For compound 9-3, MS: 414[M+H]⁺

### Step 3: Synthesis of Compound 9-4

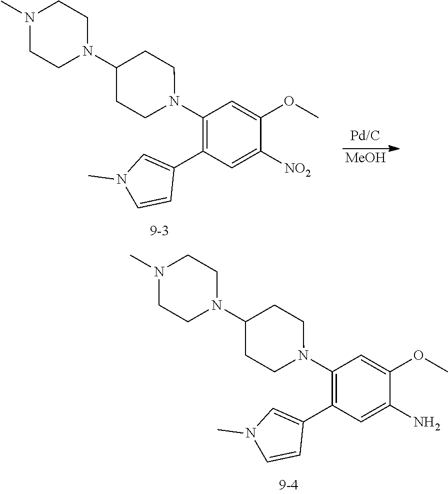

The compound 9-4 was synthesized as the method described for compound 1-4 using compound 9-3 instead of raw compound 1-3. For compound 9-4, MS: 384[M+H]⁺

### Step 4: Synthesis of Compound 9

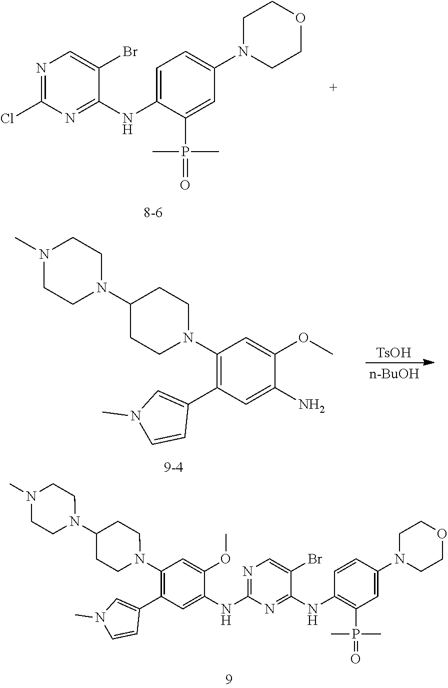

The compound 9 was synthesized as the method described for compound 1 using compound 9-4 instead of raw compound 1-4. For compound 9, MS: 747[M+H]⁺

### EXAMPLE 10

### Synthesis of Compound 10

### (2-((5-Bromo-2-((2-methoxy-5-methyl-4-(4-(4-methylpiperazin-1-yl)piperidin-1-yl)phenyl)Amino)pyrimidin-4-yl)amino)-5-cyclopropylphenyl)dimethylphosphine oxide

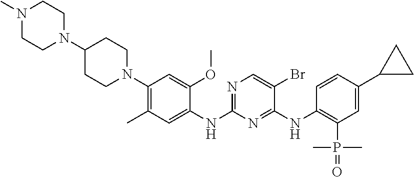 [SMILES: COc1cc(N2CCC(N3CCN(C)CC3)CC2)c(C)cc1Nc1ncc(Br)c(Nc2ccc(C3CC3)cc2P(C)(C)=O)n1]

### Step 1: Synthesis of Compound 10-2

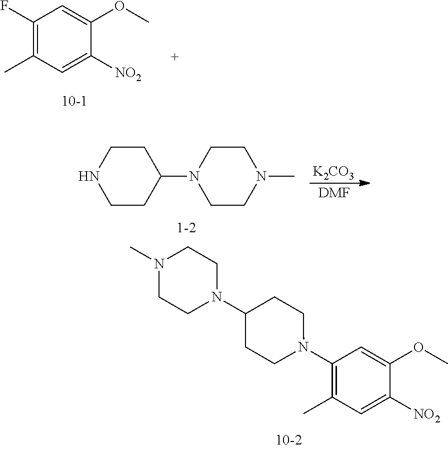

The compound 10-2 was synthesized as the method described for compound 1-3 using compound 10-1 instead of raw compound 1-1. For compound 10-2, MS: 349[M+H]⁺

### Step 2: Synthesis of Compound 10-3

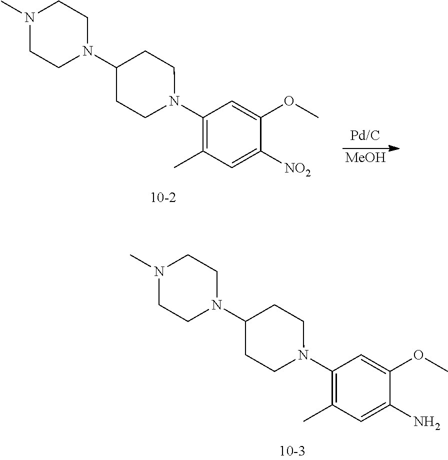

The compound 10-3 was synthesized as the method described for compound 1-4 using compound 10-2 instead of raw compound 1-3. For compound 10-3, MS: 319[M+H]⁺

### Step 3: Synthesis of Compound 11

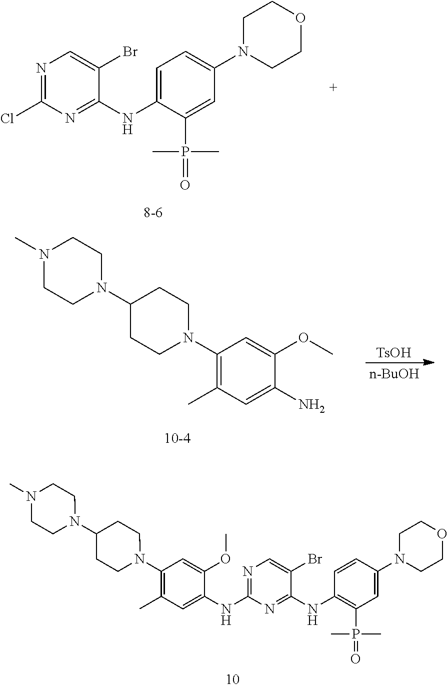

The compound 10 was synthesized as the method described for compound 1 using compound 10-4 instead of raw compound 1-4. For compound 10, MS: 682[M+H]⁺

### CONTROL EXAMPLE 1

WO2009143389 disclosed on Page 216 of the control example 1, but did not give any preparation method and effect data. This application provides a preparation method for control example 1 as follows:

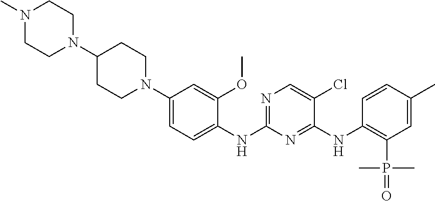

### CONTROL EXAMPLE 1

### Step 1: Synthesis of Compound 1-3

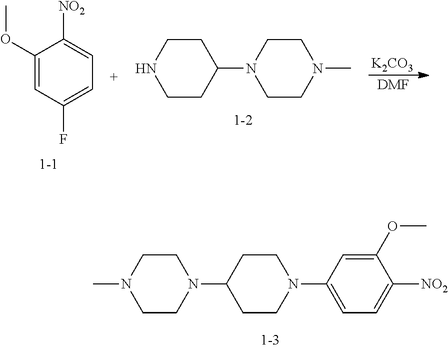

Add compounds 1-1 (1.00 g), 1-2 (1.29 g), K₂CO₃ (1.62 g) and DMSO (10 mL) successively to the reaction flask, heat to 90° C., and stir with heating for 12 hr. The reaction was complete as monitored by LCMS, and then stopped. The reaction solution was poured into water (50 mL), and extracted with DCM (2×30 mL); the organic phase was washed with water (3×20 mL) and saturated brine (20 mL), dried over anhydrous sodium sulfate, and concentrated. The resulting crude product was slurried with ether (20 mL) to obtain the yellow solid of desired product 1-3 (1.60 g), MS: 335 [M+H]⁺

### Step 2: Synthesis of Compound 1-3

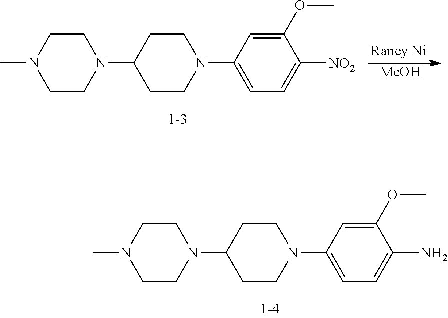 [SMILES: "MeOH" ; COc1cc(N2CCC(N3CCN(C)CC3)CC2)ccc1[N+](=O)[O-] ; COc1cc(N2CCC(N3CCN(C)CC3)CC2)ccc1N]

Add compound 1-3 (1.60 g), raney nickel (0.50 g) and MeOH (20 mL) successively to the reaction flask, introduce Hz; the reaction solution was stirred at room temperature for 3 hr. The reaction was complete as monitored by LCMS, and then stopped. The reaction solution was filtered with suction, and rinsed with methanol (20 mL); the organic phase was collected, and the solvent was removed to obtain the gray solid of desired product 1-4 (1.45 g), MS: 305 [M+H]⁺

### Step 3: Synthesis of Compound 1-7

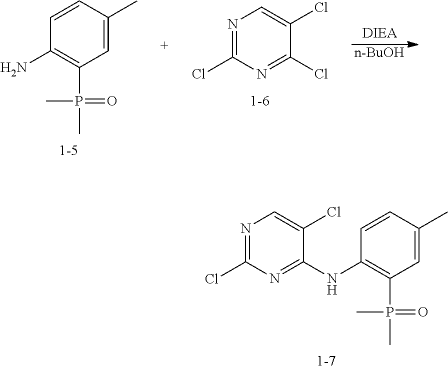

Add compounds 1-5 (0.5 g), 1-6 (0.5 g), DIEA (1.06 g) and n-butanol (5 mL) successively to the reaction flask, heat to 100° C., and stir for 3 hr. The reaction was complete as monitored by LCMS, and then stopped. The reaction solution was concentrated, separated and purified by column chromatography (dichloromethane:methanol=20:1), and the solvent was removed to obtain the white solid of desired product compound 1-7 (600 mg), MS: 330 [M+H]⁺

### Step 4: Synthesis of Control Example 1

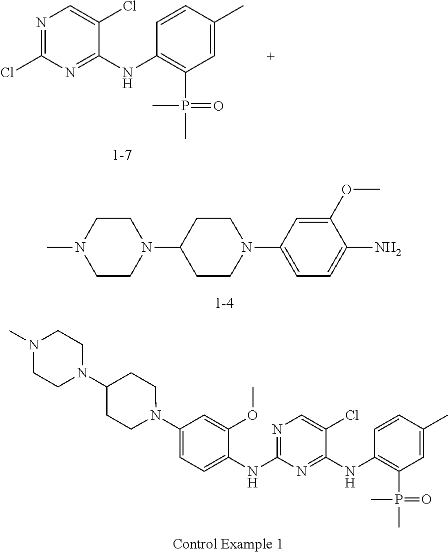

Add compounds 1-7 (100 mg), 1-4 (92 mg), p-toluenesulfonic acid (104 mg) and n-butanol (6 mL) successively to the reaction flask, heat to 100° C., and stir for 5 hr. The reaction was complete as monitored by LCMS, and then stopped. The reaction solution was poured into sodium carbonate solution (15 mL), and extracted with dichloromethane (2×15 mL); the organic phase was washed with saturated brine (3×10 mL), dried over anhydrous sodium sulfate, separated and purified by column chromatography (dichloromethane:methanol=10:1), and then concentrated to obtain off-white solid of control example 1 (63 mg), MS: 598 [M+H]⁺

### Pharmacological Test

Test 1 EGFR Δ19del/T790M/C797S Kinase Test

Mobility variation analysis was performed to determine the affinity of the compound for EGFRΔ19del/T790M/C797S. The enzymatic reaction scheme is as follows:

1. Prepare 1*kinase buffer as follows.

| 1*kinase buffer | Final concentration |
|---|---|
| HEPES PH 7.5(mM) | 50 |
| Brij-35 | 0.0150% |
| DTT(mM) | 2 |
| Mgcl₂, Mncl₂ (mM) | 10 |

2. Prepare compound concentration gradient: The test compound was tested at concentration of 300 nM, diluted with 100% DMSO solution in a 96-well plate to 100-fold final concentration, and then diluted to 3-fold concentration with Precision.10. Compound at each concentration was further diluted to 5-fold final concentration of intermediate dilution.

3. Add 5 μL of each prepared intermediate dilution compounds to the compound wells of 384-well plate respectively, and test the repeated wells of each concentration; add 5 μL of 5% DMSO into the negative control well and the positive control well respectively.

4. Prepare 2.5-fold final concentration of kinase solution with 1×Kinase buffer.

5. Add 10 μL of 2.5-fold final concentration of kinase solution to the compound well and the positive control well; add 10 μL of 1×Kinase buffer to the negative control well.

6. Centrifuge at 1000 rpm for 30 s, shake the reaction plate for well mixing, and then incubate at room temperature for 10 minutes.

7. Prepare a mixture of ATP and Kinase substrate (5-FAM-EEPLYWSFPAKKK-CONH₂) at 2.5-fold final concentration with 1×Kinase buffer.

8. Add 10 μL mixture of ATP and substrate at 2.5-fold final concentration to start reaction.

9. Centrifuge the 384-well plate at 1000 rpm for 30 s, shake it well and then incubate it at room temperature for corresponding time.

10. Add 30 μL of stop detection solution to stop the kinase reaction, centrifuge at 1000 rpm for 30 s, and shake it well.

11. Read conversion rate with Caliper EZ Reader.

Convert the conversion rate to inhibition rate:

inhibition %=(max−conversion % sample)/(max−min)*100.

“max”: Mean value of negative control wells; “min”: Mean value of positive control wells; conversion % sample: Sample conversion reading.

GraphPad Prism 5 was used for % inhibition curve fitting to obtain the IC₅₀ value.

The calculation formula: Y=inhibition %_min+(inhibition %_max−inhibition %_min)/(1+(IC₅₀/X)\hat{ }slope). wherein, Y is inhibition %; X is concentration of compound to be tested.

The results are expressed as IC₅₀ values, as shown in Table 1.

TABLE 1

| | EGFR |
|---|---|
| No. | Δ19del/T790M/C797S IC₅₀(nM) |
| Control example 1 | 2.7 |
| 1 | 0.3 |
| 2 | 0.4 |
| 3 | 0.4 |
| 4 | 1.2 |
| 5 | 1.6 |
| 6 | 0.7 |
| 7 | 1.1 |
| 8 | 0.3 |
| 9 | 0.3 |
| 10 | 0.3 |

### Test 2 Ba/F3-Δ19del/T790M/C797S and Ba/F3-L858R/T790M/C797S Cell

### Proliferation Assay

1. Cell Culture

Cell line: Ba/F3 cells with Δ19del/T790M/C797S or L858R/T790M/C797S mutation gene stably over-expressed named Ba/F3-Δ19del/T790M/C797S and Ba/F3-L858R/T790M/C797S, and A431 wild-type cell line.

A. Culture Medium

RPMI 1640, 10% FBS and 1% PS; DMEM, 10% FBS and 1% PS

B. Cell Recovery

a) The medium was preheated in a 37° C. water bath in advance.

b) Remove the cryogenic vials from the liquid nitrogen tank, quickly put it into a 37° C. water bath, and completely melt it in 1 min.

c) Transfer the cell suspension to a 15 mL centrifuge tube containing 8 mL of medium, and centrifuge at 1000 rpm for 5 min.

d) Discard the supernatant, resuspend the cells in 1 mL of culture medium, transfer it to a 75 cm² flask containing 15 mL of culture medium, and culture the cells in a incubator with 5% CO₂ at 37° C.

C. Cell Passage

a) The medium was preheated in a 37° C. water bath in advance.

b) Collect the cells in a 15 mL centrifuge tube and centrifuge at 1000 rpm for 5 min. Discard the supernatant, count to make the cell density at 1×10⁴ cells/mL, and then place it in a incubator with 5% CO2 at 37° C.

2. Compound Preparation

a) Dilute the test compound (20 mM stock solution) to 10 mM with 100% DMSO as the starting concentration, and then serially dilute 3 times with a “9+0” concentration in 96-well dilution plate (Cat #P-05525, Labcyte);

b) Dilute compound solution thereof to 1:100 in medium to prepare 10-fold working solution.

3. Cell Plate Culture

a) Centrifuge the growth cells in logarithmic phase at 1000 rpm for 5 minutes, resuspend the cells in culture medium, and then count the cells;

b) Inoculate the cells into a 96-well cell culture plate with a density of 2000 cells/well;

4. Compound Treatment

a) 15 μl of compounds prepared at step 2 were added to cell plate, the final concentrations were 1000, 333, 111.1, 370.4, 123.5, 41.2, 13.7, 4.6, 1.5 and 0 nM, and the final concentration of DMSO was 0.1%. The blank control well was a culture medium (0.1% DMSO);

b) Incubate cells in an incubator for another 72 hours;

5. Assay

a) Take out the 96-well cell culture plate and add 50 μl of CTG reagent (CellTiter Glo kit, promega, Cat#G7573);

b) Shake the plate for 2 minutes and let cool at room temperature for 10 minutes;

c) Read luminous signal value with PerkinElmer reader.

Analysis of Test Data

The data were analyzed by GraphPad Prism 6.0 software to obtain the fitting curve of compound activity.

Fitting compound IC₅₀ from nonlinear regression equation:

Y=Min+(Max−Min)/(1+10\hat{ }((Log IC50−X)*slope));

X: Logarithm of compound concentration; Y: luminous signal value.

The result of cell proliferation assay is expressed by IC50, as shown in Table 2.

TABLE 2

| | BaF3 | A431 |
|---|---|---|
| No. | Δ19del/T790M/C797SIC₅₀(nM) | IC₅₀(nM) |
| Control example 1 | 49.9 | 597.5 |
| 1 | 1.2 | 681.8 |
| 2 | 7.2 | 267.7 |
| 3 | 55.4 | 830 |
| 4 | / | / |
| 5 | 302 | / |
| 6 | 15.6 | 674.4 |
| 7 | 38.3 | 1405 |
| 8 | 30.4 | 886 |
| 9 | 5.2 | 604 |
| 10 | 9.7 | 543.2 |

Notes:
“—” stands for “not tested”.

### Test 3 Pharmacokinetic Experiment

Male SD rats (3 rats in each group) were administered orally, and fasted overnight from at least 12 hours before administration to 4 hours after administration before the experiment. Blood was taken from the orbital vein. The time points of blood collection for oral administration were: 15 min, 30 min, 1 hr, 2 hr, 4 hr, 7 hr and 24 hr, the dosage of administration was 5 mpk, and the blood collection volume was 300 μL. After anticoagulant treatment with 2.0% EDTA, the blood was centrifuged at 4000 rpm for 5 min, and then about 100 μL was taken and placed for testing at −20° C. Plasma samples were analyzed by liquid chromatography tandem mass spectrometry (LC-MS/MS). The plasma concentration-time data of individual animals were analyzed by the non-compartment model of WinNonlin (V4.1, Pharsight) software, and the pharmacokinetic parameters of the compounds tested were calculated. The PK characteristics of compounds in rats are shown in Table 3.

TABLE 3

| | | Cₘₐₓ | AUCₗₐₛₜ |
|---|---|---|---|
| Compound | t_{½} (h) | (ng/mL) | (h · ng/mL) |
| 1 | 9.1 | 2100 | 32921 |

1. A compound of Formula I, or a stereoisomer, tautomer, deuterated compound, pharmaceutically acceptable salt, prodrug, chelate, non-covalent complex or solvate thereof, 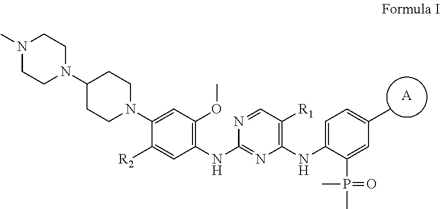
wherein,
R₁ is halogen, —C₁₋₆ alkyl or —C₁₋₆ alkoxy;
R₂ is selected from H, —C₁₋₆ alkyl, halogen or —C₅₋₆ heteroaryl, wherein the heteroatom of —C₅₋₆ heteroaryl consists of one or two N, O, S atoms, and can be substituted with —C₁₋₆ alkyl;
ring A is selected from —C₃₋₆ saturated carbocycle or —C₃₋₆ saturated heterocycle, wherein the heteroatom of —C₃₋₆ saturated heterocycle consists of one or two N, O, S atoms.

2. The compound of claim 1, wherein R₁ is selected from Cl, Br or —OCH₃.

3. The compound of claim 2, wherein R₁ is selected from Cl or Br.

4. The compound of claim 1, wherein R₂ is selected from H, —CH₃, —CH₂CH₃,

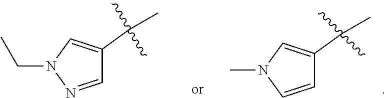

5. The compound of claim 4, wherein R₂ is selected from —CH₂CH₃ or

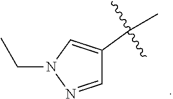 [SMILES: *c1cnn(CC)c1]

6. The compound of claim 1, wherein R₁ is Br, and R₂ is —CH₂CH₃.

7. The compound of claim 1, wherein ring A is a —C₃₋₆ saturated carbocycle.

8. The compound of claim 7, wherein ring A is selected from

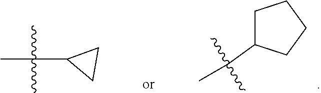

9. The compound of claim 1, wherein ring A is a —C₃₋₆ saturated heterocycle.

10. The compound of claim 9, wherein ring A is selected from

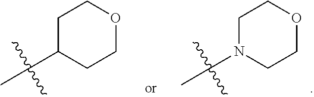 [SMILES: *C1CCOCC1 ; *N1CCOCC1]

11. The compound of claim 1, wherein the compound is:
1) (2-((5-bromo-2-((5-ethyl-2-methoxy-4-(4-(4-methylpiperazin-1-yl)piperidin-1-yl)phenyl)amino)pyrimidin-4-yl)amino)-5-cyclopropylphenyl)dimethylphosphine oxide;
2) (2-((5-bromo-2-((5-(1-ethyl-1H-pyrazol-4-yl)-2-methoxy-4-(4-(4-methylpiperazin-1-yl)piperidin-1-yl)phenyl)amino)pyrimidin-4-yl)amino)-5-cyclopropylphenyl)dimethylphosphine oxide;
3) (5-cyclopropyl-2-((2-((5-(1-ethyl-1H-pyrazol -4-yl)-2-methoxy-4-(4-(4-methylpiperazin-1-yl)piperidin-1-yl)phenyl)amino)-5-methoxypyrimidin-4-yl)amino)phenyl)dimethylphosphine oxide;
4) (2-((5-chloro-2-((2-methoxy-4-(4-(4-methylpiperazin-1-yl)piperazin-1-yl)phenyl)amino)pyrimidin-4-yl)amino)-5-cyclopropylphenyl)dimethylphosphine oxide;
5) (5-propyl-2-((2-((5-ethyl-2-methoxy-4-(4-(4-methylpiperazin-1-yl)piperidin-1-yl)phenyl)amino)-5-methoxypyrimidin-4-yl)amino)phenyl)dimethylphosphine oxide;
6) (2-((5-bromo-2-((5-ethyl-2-methoxy-4-(4-(4-methylpiperazin-1-yl)piperidin-1-yl)phenyl)amino)pyrimidin-4-yl)amino)-5-(tetrahydro-2H-pyran-4-yl)phenyl)dimethylphosphine oxide;
7) (2-((5-chloro-2-((5-ethyl-2-methoxy-4-(4-(4-methylpiperazin-1-yl)piperidin-1-yl)phenyl)amino)pyrimidin-4-yl)amino)-5-cyclopentylphenyl)dimethylphosphine oxide;
8) (2-((5-bromo-2-((5-ethyl-2-methoxy-4-(4-(4-methylpiperazin-1-yl)piperidin-1-yl)phenyl)amino)pyrimidin-4-yl)amino-5-morpholinyl)dimethylphosphine oxide;
9) (2-((5-bromo-2-((2-methoxy-5-(1-methyl-1H-pyrrol-3-yl)-4-(4-(4-methylpiperazin-1-yl)piperidin-1-yl)phenyl)amino)pyrimidin-4-yl)-amino)-5-cyclopropylphenyl)dimethylphosphine oxide; or
10) (2-((5-bromo-2-((2-methoxy-5-methyl-4-(4-(4-methylpiperazin-1-yl)piperidin-1-yl)phenyl)amino)pyrimidin-4-yl)amino)-5-cyclopropylphenyl)dimethylphosphine oxide.

12. A pharmaceutical composition comprising a compound of claim 1, or a pharmaceutically acceptable salt or stereoisomer thereof, and at least one pharmaceutically acceptable carrier or adjuvant.

13. A method for inhibiting various forms of EGFR, wherein the various forms of EGFR are mutant forms of EGFR, including L858R, Δ19del, T790M and C797S, and any combinations thereof, said method comprising administering to a patient a compound of claim 1, or a pharmaceutically acceptable salt thereof.

14. A method for treating EGFR-driven cancer, said method comprising administering to a patient in need thereof a therapeutically effective amount of a compound of claim 1, or pharmaceutically acceptable salts thereof.

15. The method of claim 14, wherein the EGFR-driven cancer is characterized by the presence of one or more mutations selected from: (i) C797S, (ii) L858R and C797S, (iii) C797S and T790M, (iv) L858R, T790M and C797S, or (v) Δ19del, T790M and C797S.

16. The method of claim 14, wherein the EGFR-driven cancer comprises colon cancer, stomach cancer, thyroid cancer, lung cancer, leukemia, pancreatic cancer, melanoma, brain cancer, kidney cancer, prostate cancer, ovarian cancer, or breast cancer.

17. The method of claim 16, wherein the lung cancer is a non-small cell lung cancer caused by EGFR^{L858R/T790M/C797S} or EGFR^{Δ19del/T790M/C797S} mutant.

18. (canceled)

19. (canceled)

20. (canceled)

21. (canceled)
